(19) United States
(12) Patent Application Publication
(10) Pub. No.: US 2012/0271085 A1
(43) Pub. Date: Oct. 25, 2012

(54) METHOD FOR PRODUCING DISTILLATE FROM A HYDROCARBON FEED, COMPRISING ALCOHOL CONDENSATION

(75) Inventors: Nikolai Nesterenko, Nivelles (BE); Delphine Minoux, Nivelles (BE); Sander Van Donk, Sainte Adresse (FR); Jean-Pierre Dath, Beloeil Hainaut (BE)

(73) Assignee: TOTAL RAFFINAGE MARKETING, Puteaux (FR)

(21) Appl. No.: 13/501,382

(22) PCT Filed: Oct. 13, 2010
(86) PCT No.: PCT/FR10/52168

§ 371 Date: Jun. 29, 2012

(30) Foreign Application Priority Data
Oct. 13, 2009 (FR) 09 57160

(51) Int. Cl. C07C 1/20

(52) U.S. Cl. 585/324

(57) ABSTRACT

The invention relates to a method for converting a hydrocarbon feed containing C3-C10 olefins into a distillate, whereby the quantities of olefins having a chain length that is too short can be reduced in order to be exploited (typically C10 or even less) and the C10+ molecule yields can be increased, while controlling the exothermicity of the oligomerisation reactions. This effect is obtained by oligomerising the hydrocarbon feed in the presence of at least one part of the products resulting from the pre-conversion, by means of condensation, of light oxygen molecules (comprising at least one alcohol having at least two carbon atoms) which can originate from biomass.

The invention relates to a process for producing distillate by oligomerization starting with a hydrocarbon-based charge comprising a condensation of an alcohol comprising at least two carbon atoms. In particular, it is a process for producing distillate by oligomerization of a hydrocarbon-based charge and of at least one light organic oxygenated compound, comprising a step of preconversion of light oxygenated compounds by condensation.

More particularly, the invention relates to a process for converting a charge rich in C3-C10 olefins into a product rich in distillates, especially middle distillates.

The term “distillate” means hydrocarbons containing 10 or more carbon atoms, middle distillates comprising from 10 to 20 carbon atoms and distilling in the temperature range from 145° C. to 350° C. Among the distillates, C10-C12 olefins (jet fuel) and C12+ olefins (diesel) will especially be distinguished.

Usually, charges rich in C3-C10 olefins are treated via catalytic oligomerization processes in order to increase the length of the olefin chains. The product obtained then undergoes a fractional distillation in order to separate the gasoline (C5-C9), the jet fuel (C10-C12) and the diesel (C12+).

The majority of the processes described in the bibliography propose solutions for oligomerizing a charge rich in olefins and more particularly in C3-C4 olefins. These olefins may be converted into oligomers under relatively mild conditions in which the contribution of the cracking reaction is negligible. The effect of the presence in the charge of a broader range of olefins in combination with an amount of inert or semi-inert material (paraffins, naphthenes and aromatics) makes the application of standard oligomerization processes less efficient.

Due to the different reactivity of C3-C10 olefins and above all due to the lower reactivity of C5-C10 olefins, it may be necessary to work at a higher temperature in order to improve the direct yield in an oligomerization process. Working at a higher temperature may lead to the cracking and aromatization of already-formed oligomers from more reactive olefins as long as part of the olefins of the charge still remains unconverted. In this case, the products of the C3-C10 olefin oligomerization processes contain large amounts of olefins bearing an excessively short chain length (less than 10 carbon atoms). These olefins cannot be used directly and must be recycled into the process in order to increase their chain length. This recycling, which may be up to 75% of the effluent produced, increases the complexity and costs of the installation. In addition, the recycling requires an additional step in the separation of olefins with a short chain length of the corresponding paraffins. The fact that these molecules have similar IPBs (initial distillation points) does not make it possible to use distillation, and necessitates the use of more sophisticated separation processes.

There is thus a need to reduce, or even eliminate, these recycling operations, by increasing the yield of distillates, especially of C10-C20 middle distillates.

The present invention provides a solution for improving the process for the oligomerization of olefinic charge containing a large amount of inert material (paraffins, naphthenes and aromatics) and olefins with different reactivity.

The presence of an oxygenated molecule as water precursor leads to moderation of the acidity of the oligomerization catalyst, limiting the cracking reaction, and gives a solution for managing the exothermicity of the oligomerization reaction.

On account of a lower reactivity of heavy olefins relative to light olefins in oligomerization, the presence of an oxygenated molecule as precursor for a light olefin generated progressively may lead to a high conversion of the heavy olefins, resulting in a product whose chain length is longer.

Oxygenated compounds comprising one or two carbon atoms (alcohols, aldehydes and the corresponding ethers, including DEE) in their structure are less expensive and more available than C3+ oxygenated compounds (propanol, butanol, etc.). However, the presence of ethanol or DEE in the oligomerization reaction mixture may lead to rapid deactivation of an acidic catalyst. In addition, the incorporation of ethylene generated in the liquid effluent from ethanol on an acidic catalyst requires very different conditions compared with C3-C10 olefins. Transforming methanol into hydrocarbons is an exothermic reaction. A combination of two exothermic reactions (oligomerization and conversion of the methanol into hydrocarbons) is not always favorable. A step of preconversion of the light oxygenated compounds into heavy oxygenated compounds and hydrocarbons, via condensation on basic catalysts before oligomerization, makes it possible to use a cheap oxygenated compound for the oligomerization, which avoids the drawbacks described above.

Moreover, oligomerization reactions are highly exothermic, which requires a control of the temperature of oligomerization units. This control may be performed with a unit using a system of several reactors, with cooling devices between reactors.

Furthermore, there is currently a tendency toward encouraging the development of biofuels, especially by encouraging the incorporation of fuel or of product of agricultural origin into fuels. One of the main sources of biofuels is bioethanol derived from biomass. It is, however, currently difficult to prepare C10+ distillates, which in particular are sparingly branched, from bioethanol. The known processes require high levels of recycling, heavy investment in C2 extraction equipment, and substantial purging: a large amount of the bioethanol is thus found in the form of a light fraction with low added value.

The invention is directed toward overcoming these drawbacks by proposing a process for converting a hydrocarbon-based charge containing C3-C10 olefins into distillate, making it possible to reduce the amounts of olefins whose chain length is too short to be exploited (typically C10, or even less) and to increase the yields of C10+ molecules, while at the same time controlling the exothermicity of the oligomerization reactions. This effect is obtained by performing the oligomerization of the hydrocarbon-based charge in the presence of at least part of the products derived from the preconversion of light oxygenated molecules (comprising at least one alcohol comprising at least two carbon atoms, for example ethanol) by condensation, these molecules possibly originating from biomass.

The invention thus allows an appreciable reduction in recycling, or even suppression thereof.

The alcohol may be subjected alone to the condensation or in the presence of methanol, DME, DEE, formaldehyde, acetaldehyde or ethylene glycol (oxygenated molecules containing one or two carbon atoms).

To this end, a first subject of the invention concerns a process for producing distillate, especially middle distillates, by conversion of a hydrocarbon-based charge containing C3-C10 olefins, comprising a first step of preconversion of light oxygenated molecules by condensation, these light oxygenated molecules comprising at least one alcohol comprising at least two carbon atoms, and a second step of oligomerization, in which the hydrocarbon-based charge is oligomerized in at least one oligomerization reactor in the presence of at least part of the effluent derived from the first step.

The term “light oxygenated molecules” means molecules comprising one to four carbon atoms.

Thus, to perform the first preconversion step, at least one light oxygenated molecule consisting of an alcohol comprising at least two carbon atoms will be chosen, which may be mixed with other oxygenated molecules comprising one or two carbon atoms.

In particular, when the hydrocarbon-based charge is oligomerized in an installation comprising several reactors in series, the presence of part of the effluent derived from the first step is not obligatory at the inlet of the first reactor. The condensation effluent may be injected into the middle of the first reactor and/or into the inlet of the second reactor, for example.

Preferably, the total weight ratio between the effluent derived from the first step injected into the oligomerization installation and the hydrocarbon-based charge is from 0.0001 to 1000 and preferably from 0.005 to 100.

The effluent obtained derived from the condensation may then be conveyed into a separation zone in order to extract therefrom part of the water, ethylene, ethanol, and other unconverted light oxygenated molecules.

Advantageously, the effluent derived from the condensation is treated by selective hydrogenation in order to convert the diolefins, more particularly butadienes, into the corresponding olefins.

The alcohol comprising at least two carbon atoms, for example ethanol, used in the first step may especially be derived from biomass, for example by alcohol fermentation or via the synthesis gas route.

Preferably, the first step of preconversion of oxygenated molecules via condensation comprising an alcohol containing at least two carbon atoms, such as ethanol, especially produces propanol, isobutanol, n-butanol, heavier alcohols, and corresponding olefins.

It has been discovered that the oligomerization of a C3-C10 hydrocarbon-based charge in the presence of an effluent derived from this condensation makes it possible to increase the yield of C10+ distillates.

Furthermore, in the second oligomerization step, the incorporation of C3+ alcohols into the oligomerization reaction products takes place via the dehydration reactions of alcohols to the corresponding olefins. Since these dehydration reactions are endothermic, the thermal equilibrium for the oligomerization process is thus improved, the exothermicity of the oligomerization reactions supplying the energy required for the dehydration of the alcohols present. It is thus easier to manage the exothermicity of the oligomerization, which makes it possible to optimize the conversions of each reaction zone and to reduce the total energy expenditure, and similarly the corresponding investment costs.

It may be envisioned that the effluent derived from the first step, after the selective hydrogenation, be totally or partly added to the hydrocarbon-based charge in the second step. For example, the weight ratio of the effluent derived from the first step and of the hydrocarbon-based charge may be from 0.0001 to 1000 and preferably from 0.005 to 100.

First Step: Condensation of the Alcohol

Advantageously, the first condensation step comprises the placing in contact of the light oxygenated molecules in aqueous phase, comprising at least one alcohol comprising at least two carbon atoms, for example ethanol, with at least one basic catalyst, at a temperature and a pressure that are sufficient to obtain a liquid effluent containing at least 40% by weight and preferably at least 50% by weight of molecules containing three or more carbon atoms, and less than 10% by weight of ethylene.

The remaining effluent comprises unconverted oxygenated molecules. These molecules are either recycled into the condensation reactor, or they are supplied for the oligomerization with the remaining effluent.

The alcohol, for example ethanol, may be subjected alone to the condensation or in the presence of methanol, DME, DEE, formaldehyde, acetaldehyde or ethylene glycol (oxygenated molecules with one or two carbon atoms), or mixtures thereof.

The alcohol, for example ethanol, in aqueous phase may optionally be diluted with an inert gas such as N₂ or CO₂.

The production of heavy alcohols (three or more carbon atoms) from light alcohols (1-2 carbon atoms) and more particularly of 1-butanol from ethanol over a basic catalyst is known in the literature as the “Guerbet reaction”.

Guerbet syntheses have typically been used to prepare branched-chain alcohols of higher molecular weight from starting materials of lower molecular weight. An example of a Guerbet synthesis, consisting of a dimerization of an alcohol, is described in document U.S. Pat. No. 4,518,810. Another example of a Guerbet synthesis, consisting of the condensation of two different alcohols, is described in document U.S. Pat. No. 2,050,788.

The main side products of the condensation of ethanol are 1-butene, 1,3-butadiene, heavy alcohols (5 or more carbon atoms), traces of ethylene, unconverted ethanol, aldehydes and water.

Ethylene and the other light gases, such as ethane, may then be easily separated from the hydrocarbons and the heavier oxygenated compounds (5 or more carbon atoms) forming the liquid phase, for example by flash separation. Any excess water may then be removed.

Before being placed in contact with the hydrocarbon-based charge during the oligomerization step, the effluent from the first step may undergo a selective hydrogenation of the aldehydes to alcohols, especially as heavy alcohols, and of the dienes to olefins.

This selective hydrogenation will preferably be performed before the separation.

During the oligomerization, the heavy alcohols (5 or more carbon atoms) undergo a dehydration to the corresponding olefins. Thus, the condensation side products form olefins or olefin precursors, which improve the quality of the hydrocarbon-based charge to be oligomerized by increasing its olefin content.

Advantageously, the first step may be performed at a temperature of from 150 to 500° C. and a pressure from 0.1 to 40 MPa.

A basic catalyst that is suitable for the first step of the present process will be either a Brønsted base, which has the capacity of accepting protons, or a Lewis base, which has a lone pair of electrons with which it may form a covalent bond with an atom, a molecule or an ion.

Preferentially, the catalyst will contain at least two metal oxides, chosen from oxides of metals of groups II and III (including the lanthanide and actinide series) and group IV. These metal oxides may be combined in different ways to produce the active mixed metal oxide.

These active mixed metal oxides may be prepared according to very different methods, preferentially from precursor salts of metals from group II, from group III and/or from group IV. However, any other source of metal oxide (such as oxychlorides, alkoxides or nitrates) is also included.

A first method for preparing this mixed metal oxide consists in impregnating the precursor of the metal oxide onto an already preformed metal oxide, the latter having possibly undergone a prior hydrothermal treatment. The resulting solid is then dried and then calcined preferably under an oxidizing atmosphere, at a minimum temperature of 400° C., for example from 600 to 900° C., typically from 650 to 800° C., to form the active mixed metal oxide. The calcination time is from 0 to 48 hours, more precisely from 0.5 to 24 hours, for example from 1 to 10 hours. In the context of the subject of this invention, the calcination will be performed at 700° C., for 1 to 3 hours.

A second method for preparing this mixed metal oxide may be performed by combining a first liquid solution, which is preferably aqueous, containing the precursor of the metal from group II, III or IV, with a second solution, which is preferably aqueous, containing the precursor of the metal from another group (II, III or IV). The mixing of these two solutions takes place under conditions giving rise to coprecipitation of the hydrated precursor of the mixed metal oxide. The addition of a precipitation agent is occasionally required. Such a precipitation agent will be sodium hydroxide or ammonium hydroxide. The coprecipitation temperature chosen is less than 200° C., preferably from 20 to 200° C. The resulting gel then undergoes a hydrothermal treatment in a sealed cylinder under the following conditions: a pressure above atmospheric pressure, at a temperature above 100° C., for a time of 20 days, which may be as short as 3 days.

The hydrated precursor of the mixed metal oxide recovered either by filtration or centrifugation is washed and dried. The solid is then calcined preferably under an oxidizing atmosphere, at a minimum temperature of 400° C., for example from 600 to 900° C., typically from 650 to 800° C. to form the active mixed metal oxide. The calcination time is from 0 to 48 hours, more precisely from 0.5 to 24 hours, for example from 1 to 10 hours. In the context of the invention, the calcination was performed at 700° C., from 1 to 3 hours.

As other types of suitable basic catalysts, hydroxides, carbonates, silicates, phosphates and aluminates, and all combinations, will be included without limitation. The compounds constituting the abovementioned suitable catalysts will preferably be selected from groups 1, group 2 and rare earths of the Periodic Table. Cesium, rubidium, magnesium, lithium, barium, potassium and lanthanum will preferably be chosen.

Other catalysts that may be used are mixed metal oxide catalysts having, besides basic properties, redox properties under the operating conditions employed for the conversion of the oxygenated compounds into olefinic products. Typically, the catalyst will comprise at least one transition metal oxide, preferably two, generally from period 4, 5 or 6 of the Periodic Table, preference being given to the transition metals Ag, Cu, Ce, Zn, Cd, Ti, V, Cr, Mo, Fe, Co and Ni. However, other metal oxides may be used in the composition of the mixed oxides, for instance oxides of metals from group IV including zirconium, those of group I (alkali metals) including sodium, potassium and cesium, and those of group II (alkaline-earth metals) more specifically including magnesium, calcium or barium. The oxides of rare-earth metals (scandium, yttrium, lanthanum and also those of the lanthanide series (for example cerium)) are also included, as are thorium oxide from the actinide series. The redox nature of these mixed metal oxides under the operating conditions used is explained by the existence of multiple valency states of the metal, which is a well-known characteristic of the majority of the transition metals. It is the operating conditions prevailing in the process that condition the valency states of the mixed-valency metal constituting the mixed metal oxide. Thus, it is not impossible that, under the operating conditions prevailing in the process, the valency states of the metal constituting the mixed metal oxide described above vary according to the place and according to the moment under consideration in the process.

The operating conditions of the process (temperature, pressure, catalyst/charge ratio or WHSV) are described in the following paragraphs. On the other hand, the operating conditions necessary for the redox nature to intervene are much more difficult to define with only one parameter or even with a simple combination of parameters.

The catalyst suitable for the first step of the process according to the invention has a CO₂ adsorption capacity at 100° C. of at least 0.03 mg/m²: although the upper limit of the CO₂ adsorption capacity is not critical, this type of catalyst has a CO₂ adsorption capacity at 100° C. of less than or equal to 10 mg/m². Typically, the catalyst that is suitable for the first step of the invention has a CO₂ adsorption capacity at 100° C. of from 0.01 to 1 mg/m².

In terms of specific surface area (measured according to ASTM method D3663), the suitable catalysts have a specific surface area of at least 10 m²/g and more specifically a surface area of from 20 to 250 m²/g. Typically, the catalyst of the invention has a specific surface area of between 25 and 280 m²/g.

A basic catalyst that is suitable for the reaction described above may also be supported on a support, as is commonly performed by a person skilled in the art. Supports that may be used, without these examples being limiting, include alumina, titanium, silica, zirconia, zeolites, charcoal, clays, lamellar hydroxides, hydrotalcites, and all combinations.

2nd Step: Oligomerization

The hydrocarbon-based charge used may be a mixture of hydrocarbon-based effluents containing C3-C10 olefins derived from refinery or petrochemistry processes (FCC, vapor cracking, etc.). The charge may be a mixture of fractions comprising C3 FCC, C4 FCC, LCCS, LCCCS, Pygas, LCN, and mixtures, such that the content of linear olefins in the C5-fraction (C3-C5 hydrocarbons) relative to the total C3-C10 charge is less than or equal to 40% by weight.

The total olefin content in the C5-(C3-C5) fraction relative to the total C3-C10 charge supplied for the oligomerization may be greater than 40% by weight if the isoolefins are present in an amount of at least 0.5% by weight.

The total content of linear olefins may be greater than 40% by weight relative to the total charge of C3-C10 if the linear C6+ olefins (C6, C7, C8, C9, C10) are present in an amount of at least 0.5% by weight.

This charge may especially contain olefins, paraffins and aromatic compounds in all proportions, in conformity with the rules described above.

The hydrocarbon-based charge will preferably contain a small amount of dienes and of acetylenic hydrocarbons, especially less than 100 ppm of diene, preferably less than 10 ppm of C3-C5 dienes. To this end, the hydrocarbon-based charge will be treated, for example, by selective hydrogenation optionally combined with adsorption techniques.

The hydrocarbon-based charge will preferably contain a small amount of metals, for example less than 50 ppm and preferably less than 10 ppm. To this end, the hydrocarbon-based charge will be treated, for example, by selective hydrogenation optionally combined with adsorption techniques.

Advantageously, the charge used has undergone a partial extraction of the isoolefins it contains, for example by treatment in an etherification unit, thus allowing concentration as linear olefins.

In general, commercially available olefinic charges bring about deactivation of the oligomerization catalyst that is faster than expected. Although the reasons for such a deactivation are not clearly understood, it is considered that the presence of certain sulfur compounds is at least partly responsible for this drop in activity and selectivity. In particular, it would appear that aliphatic thiols, sulfides and disulfides of low molecular weight are more particularly troublesome.

It is thus established that the acceptable sulfur content in a charge of an oligomerization process must be low enough for the activity of the catalyst used not to be inhibited. In general, the sulfur content is less than or equal to 100 ppm, preferably less than or equal to 10 ppm, or even less than or equal to 1 ppm.

The removal of these sulfur compounds requires hydrotreatment steps that increase the total cost of the process, and which may lead to a reduction in the amount of olefins. This loss may prove to be very penalizing for C5-C10 fractions typically containing from 200 to 400 ppm of sulfur.

There is thus also a need to develop an oligomerization process that allows the treatment of commercially available olefinic charges without a severe prior hydrotreatment.

It is common practice to add water to the charge of a catalytic oligomerization process. This addition of water makes it possible especially to control the temperature of the oligomerization reactor, in particular during the startup of the reactor, when the catalyst is fresh and the exothermicity is greatest. The presence of water-precursor oxygenated compounds in the charge used for the process according to the invention has the advantage of increasing the sulfur tolerance of the oligomerization catalysts. The lifetime of the catalyst may thus be increased. As a result of the contents of oxygenated compounds used, the water formed during the oligomerization represents more than 0.25% by weight of the hydrocarbon-based charge.

By way of example, in order to prevent the catalytic activity of the catalyst from being substantially inhibited, the nitrogen content of the hydrocarbon-based charge is not greater than 1 ppm by weight (calculated on an atomic basis), preferably not greater than 0.5 ppm and more preferably 0.3 ppm. Furthermore, by way of example, the chloride content of the hydrocarbon-based charge is not greater than 0.5 ppm by weight (calculated on an atomic basis), preferably not greater than 0.4 ppm and more preferably 0.1 ppm.

To this end, the hydrocarbon-based charge used may have undergone a prior treatment, for example a partial hydrotreatment, a selective hydrogenation and/or a selective adsorption.

Moreover, the presence of effluents issued from the condensation in the charge of the oligomerization step increases the partial pressure of olefins, which makes it possible to improve the yield for the oligomerization process.

A first family of catalysts that can be used may comprise an acidic catalyst either of amorphous or crystalline aluminosilicate type, or a silicoaluminophosphate, in H+ form, chosen from the following list and optionally containing alkali metals or alkaline-earth metals:

MFI (ZSM-5, silicalite-1, boralite C, TS-1), MEL (ZSM-11, silicalite-2, boralite D, TS-2, SSZ-46), ASA (amorphous silica-alumina), MSA (mesoporous silica-alumina), FER (Ferrierite, FU-9, ZSM-35), MTT (ZSM-23), MWW (MCM-22, PSH-3, ITQ-1, MCM-49), TON (ZSM-22, Theta-1, NU-10), EUO (ZSM-50, EU-1), ZSM-48, MFS (ZSM-57), MTW, MAZ, SAPO-11, SAPO-5, FAU, LTL, BETA MOR, SAPO-40, SAPO-37, SAPO-41 and the family of microporous materials composed of silica, aluminum, oxygen and possibly boron.

Zeolite may be subjected to various treatments before use, which may be: ion exchange, modification with metals, steam treatment (steaming), acid treatments or any other dealumination method, surface passivation by deposition of silica, or any combination of the abovementioned treatments.

The content of alkali metals or rare-earth metals is from 0.05% to 10% by weight and preferentially from 0.2% to 5% by weight. Preferentially, the metals used are Mg, Ca, Ba, Sr, La and Ce, which are used alone or as a mixture.

A second family of catalysts used comprises phosphate-modified zeolites optionally containing an alkali metal or a rare-earth metal. In this case, the zeolite may be chosen from the following list:

MFI (ZSM-5, silicalite-1, boralite C, TS-1), MEL (ZSM-11, silicalite-2, boralite D, TS-2, SSZ-46), ASA (amorphous silica-alumina), MSA (mesoporous silica-alumina), FER (Ferrierite, FU-9, ZSM-35), MTT (ZSM-23), MWW (MCM-22, PSH-3, ITQ-1, MCM-49), TON (ZSM-22, Theta-1, NU-10), EUO (ZSM-50, EU-1), MFS (ZSM-57), ZSM-48, MTW, MAZ, FA U, LTL, BETA MOR.

The zeolite may be subjected to various treatments before use, which may be: ion exchange, modification with metals, steam treatment (steaming), acid treatments or any other dealumination method, surface passivation by deposition of silica, or any combination of the abovementioned treatments.

The content of alkali metals or of rare-earth metals is from 0.05% to 10% by weight and preferentially from 0.2% to 5% by weight. Preferentially, the metals used are Mg, Ca, Ba, Sr, La and Ce, which are used alone or as a mixture.

A third family of catalysts used comprises difunctional catalysts, comprising:
a support, from the following list: MFI (ZSM-5, silicalite-1, boralite C, TS-1), MEL (ZSM-11, silicalite-2, boralite D, TS-2, SSZ-46), ASA (amorphous silica-alumina), MSA (mesoporous silica-alumina), FER (Ferrierite, FU-9, ZSM-35), MTT (ZSM-23), MWW (MCM-22, PSH-3, ITQ-1, MCM-49), TON (ZSM-22, Theta-1, NU-10), EUO (ZSM-50, EU-1), MFS (ZSM-57), ZSM-48, MTW, MAZ, BETA, FAU, LTL, MOR, and microporous materials of the family ZSM-48 consisting of silicon, aluminum, oxygen and optionally boron. MFI or MEL (Si/Al>25), MCM-41, MCM-48, SBA-15, SBA-16, SiO2, Al2O3, hydrotalcite, or a mixture thereof; a metallic phase (Me) to a proportion of 0.1% by weight, the metal being selected from the following elements: Zn, Mn, Co, Ni, Ga, Fe, Ti, Zr, Ge, Sn and Cr used alone or as a mixture. These metal atoms may be inserted into the tetrahedral structure of the support via the tetrahedral unit [MeO₂]. The incorporation of this metal may be performed either by adding this metal during the synthesis of the support, or it may be incorporated after synthesis by ion exchange or impregnation, the metals then being incorporated in the form of cations, and not integrated into the structure of the support.

The zeolite may be subjected to various treatments before use, which may be: ion exchange, modification with metals, steam treatment (steaming), acid treatments or any other dealumination method, surface passivation by deposition of silica, or any combination of the abovementioned treatments.

The content of alkali metals or of rare-earth metals is from 0.05% to 10% by weight and preferentially from 0.2% to 5% by weight. Preferentially, the metals used are Mg, Ca, Ba, Sr, La and Ce, used alone or as a mixture.

The catalyst may be a mixture of the materials described previously in the three families of catalyst. In addition, the active phases may themselves also be combined with other constituents (binder, matrix) giving the final catalyst increased mechanical strength, or improved activity.

If the hydrocarbon-based charge is oligomerized in an installation comprising several reactors in series, the reactors of the series may be charged with the same catalyst or a different one.

Advantageously, the second step will be performed under a reducing atmosphere, for example under H₂.

In particular, the presence of a reducing atmosphere, and possibly of water, makes it possible to improve the stability of the catalyst used.

The second step of the process according to the invention has the advantage of being able to be performed in an existing oligomerization installation.

For example, an installation containing several reactors may be used, in which the exothermicity of the reaction may be controlled so as to avoid excessive temperatures. Preferably, the maximum temperature difference within the same reactor will not exceed 75° C.

The reactor(s) may be of the isothermal or adiabatic type with a fixed or moving bed. The oligomerization reaction may be performed continuously in one configuration comprising a series of fixed-bed reactors mounted in parallel, in which, when one or more reactors are in service, the other reactors undergo regeneration of the catalyst.

All the effluent derived from the condensation may be added to the charge before it enters the oligomerization reactor(s), or partly before it enters the oligomerization reactor(s), the remaining part being added to the oligomerization reactor(s), for example as a quench.

When several oligomerization reactors are used, the effluent may be added at the inlet or into the second reactor or subsequent reactors.

The process may be performed in one or more reactors.

Preferably, the process will be performed using two separate reactors.

The reaction conditions for the first reactor will be chosen so as to convert part of the olefinic compounds with a low carbon number (C3-C5) into intermediate olefins (C6+).

Advantageously, the first reactor will comprise a first catalytic zone and will function at high temperature, for example greater than or equal to 250° C., and at moderate pressure, for example less than 50 bar.

The second reactor will preferably operate at temperatures and pressures chosen so as to promote the oligomerization of heavy olefins to distillate. The effluent from the first reactor, comprising the unreacted olefins, the intermediate olefins, water and possibly other compounds such as paraffins and possibly a reducing gas, then undergoes an oligomerization in this second reactor comprising a second catalytic zone, which makes it possible to obtain an effluent of heavier hydrocarbons, rich in distillate.

Advantageously, the first reactor will function at a lower pressure and at a higher temperature and hourly space velocity than the second reactor.

It may optionally be envisioned to use the pressure difference between the two reactors in order to perform a flash separation step.

The second step may be performed under the conditions described below.

The mass throughput through the oligomerization reactor(s) will advantageously be sufficient to enable a relatively high conversion, without being too low, so as to avoid adverse side reactions. The hourly space velocity (weight hourly space velocity, WHSV) of the charge will be, for example, from 0.1 to 20 h⁻¹, preferably from 0.5 to 10 h⁻¹ and more preferably from 1 to 8 h⁻¹.

The temperature at the reactor inlet will advantageously be sufficient to allow a relatively high conversion, without being very high, so as to avoid adverse side reactions. The temperature at the reactor inlet will be, for example, from 150° C. to 400° C., preferably 200-350° C. and more preferably from 220 to 350° C.

The pressure across the oligomerization reactor(s) will advantageously be sufficient to allow a relatively high conversion, without being too low, so as to avoid adverse side reactions. The pressure across the reactor will be, for example, from 8 to 500 bara (0.8 to 50 MPa), preferably 10-150 bara (1 to 15 MPa) and more preferably from 14 to 49 bara (bar, absolute pressure) (1.4 to 4.9 MPa).

The effluent derived from the oligomerization step is then conveyed into a separation zone, in order to separate, for example, the fractions into an aqueous fraction, C5-C9 (gasoline), C10-C12 (jet fuel) and C12+ (diesel). The fractions C5-C9, C10-C12 and C12+ may undergo a drying step.

Thus, the invention makes it possible especially to obtain a jet fuel (C10-C12, “jet fuel”) from alcohols of plant origin.

The C1-C4 light fractions may also be separated out and sent as purge or optionally recycled with the hydrocarbon-based charge.

The fractions C10-C12 and C12+ separated from the effluent of the oligomerization process may undergo a hydrogenation in order to saturate the olefinic compounds and to hydrogenate the aromatic compounds. The product obtained has a high cetane number and excellent properties for use as a fuel of jet or diesel type, or the like.

The invention is now described with reference to the examples and the attached drawings, which are not limiting, in which drawings:

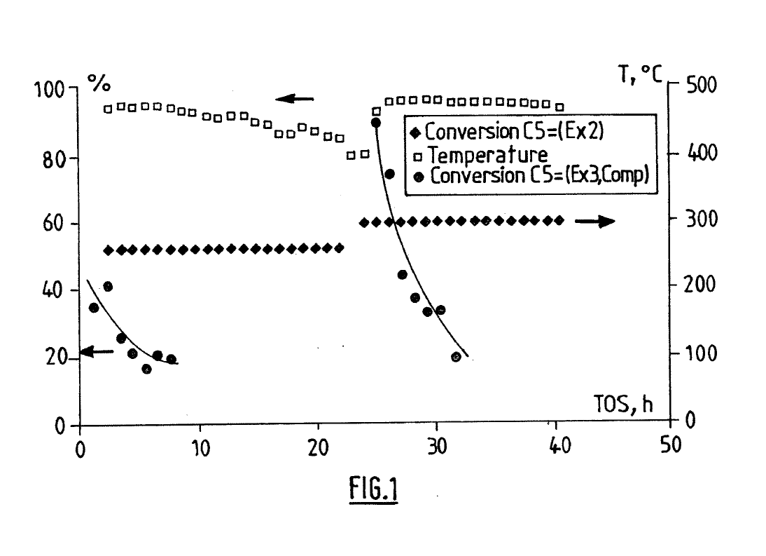

FIG. 1 represents the degree of conversion into C5 olefins of Examples 2 and 3, and also the temperature as a function of the test time (TOS);

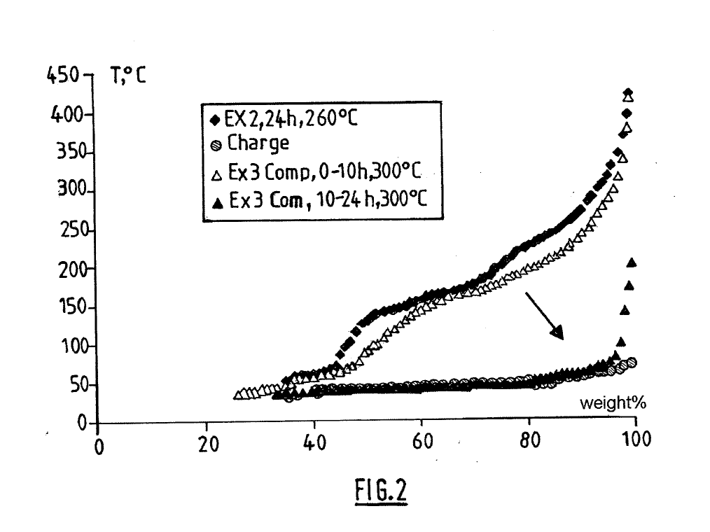

FIG. 2 represents the curves of simulated distillation of the liquid organic phases of the effluent of Example 2 (reaction temperature: 260° C.), of the charge, of Example 3 (reaction temperature: 300° C.) after 10 and 24 hours;

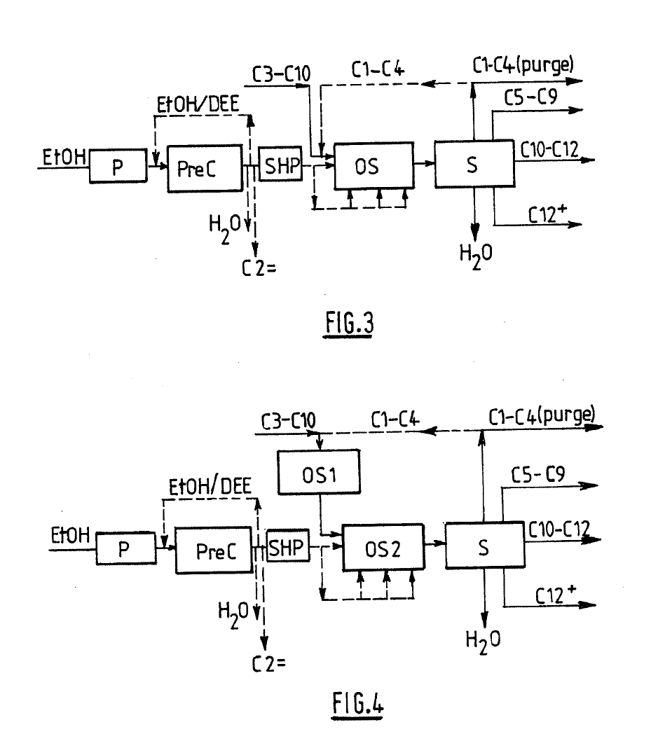

FIGS. 3 and 4 schematically represent different embodiments of the process according to the invention.

In each of the FIGS. 3 and 4:
OS represents an oligomerization zone, FIG. 4 comprising two oligomerization zones, OS et OS2,
S represents a separation zone,
SHP represents a zone of selective hydrogenation and/or of selective adsorption,
PreC represents a preconversion zone,
P represents a zone for purification of the oxygenated compounds.

In these figures, the dashed lines represent process options.

Each oligomerization zone represents, for example, an oligomerization reactor.

The scheme represented in FIG. 3 corresponds to a process in which the charge consisting of C3-C10 hydrocarbon-based compounds is treated in an oligomerization zone OS. The effluent leaving this zone OS is conveyed into the separation zone S.

In this zone S, the water is removed and the olefins are separated into C2-C4, C5-C9 (gasoline), C10-C12 (jet fuel) and diesel (C12+). Part of the C2-C4 light olefins may optionally be recycled as charge for the oligomerization zone OS.

The ethanol (optionally mixed with DDE), after having been purified in the zone P, is treated in the preconversion zone PreC. The effluent derived from the zone PreC undergoes a selective hydrogenation (SHP) before entering the oligomerization zone OS, either as a mixture with the hydrocarbon-based charge, or into the zone OS itself. The ethylene, the water and the unconverted ethanol (and possibly the unconverted DDE) may be extracted from the effluent derived from zone PreC, the unconverted ethanol (and possibly the unconverted DDE) being recycled into the zone PreC.

The scheme represented in FIG. 4 corresponds to a process that differs from the one represented in FIG. 3 only by the presence of a second oligomerization zone OS2. The C3-C10 hydrocarbon-based charge is treated in this second zone OS2 before being treated in the zone OS. In this embodiment, the C2-C4 olefins separated out in the separation zone are recycled into the second zone OS2, with the hydrocarbon-based charge.

### EXAMPLES

### Example 1

### Preparation of Catalyst A

A sample of zeolite MFI (Si/Al=82) with a crystal size of 0.2-0.3 μm supplied by Zeolyst Int. in the form of NH₄ was calcined at 550° C. for 6 hours in order to convert it into H+ form. The product thus obtained is named catalyst A.

### Example 2

### Oligomerization Test in the Presence of Butanol

20 ml (12.8 g) of catalyst A in the form of grains (35-45 mesh) were placed in a fixed-bed tubular reactor with an inside diameter of 11 mm. Before the tests, the catalyst was activated at 550° C. under a stream of nitrogen for 6 hours. After activation, the reactor was cooled to 40° C. The catalyst was placed in contact with the charge at 40° C. and at atmospheric pressure for 1 hour. Next, the pressure was increased up to the reaction value and the reactor was heated to 200° C. at a rate of 30° C./hour. The temperature was maintained for 12 hours at 200° C. and was then increased up to 260° C. (30° C./hour).

The charge used for this oligomerization test is a fraction LLCCS containing 83% by weight of C5 hydrocarbons (of which 59% by weight are olefins and 41% by weight are paraffins). The content of linear olefins in the C5 fractions is 27.2% by weight.

The mixture comprising 85% by weight of LLCCS and 15% by weight of 1-butanol was placed in contact with catalyst A under the following conditions:

Reactor inlet temperature: 260, 300° C.

Pressure P: 40 barg

Hourly space velocity (pph): 1 h⁻¹.

(P (barg)=P bar−Patm (˜1 bar))

The analysis of the products obtained was performed online by gas chromatography, the chromatograph being equipped with a capillary column.

At the reactor outlet, the gaseous phase, the liquid organic phase and the aqueous phase were separated. No recycling was performed.

The catalyst showed very little deactivation, which may be compensated for by increasing the temperature without substantially increasing the gaseous phase (FIG. 1).

The curve of simulated distillation of the liquid organic phase is reported in FIG. 2.

Table 1 below collates the degrees of conversion and the yields obtained.

TABLE 1

| | |
|---|---|
| Conversion of 1-butanol into | 99.9 |
| HC (weight %) | |
| Conversion of C5 (weight %) | >95% |

| | Relative to | |
|---|---|---|
| Yields (weight %) | carbon | Relative to the olefins |
| C1-C3 | 0.1 | 0.2 |
| nC4 | 0.5 | 0.8 |
| Total C4 | 1.2 | 1.9 |
| Gasoline (distillate <150° C.) | 57.3 | 32.4 |
| Diesel (distillate >150° C.) | 41.5 | 65.6 |

This Table 1 illustrates the possibility of producing a distillate-rich heavy hydrocarbon fraction from a charge containing a gasoline fraction and 15% butanol. The charge contained several olefins with different reactivities. The results show a virtually total conversion of butanol into hydrocarbons, little cracking (gaseous phase) despite a relatively high temperature for the oligomerization, and the conversion of a significant amount (65.6%) of olefins distilling above 150° C. from a real charge.

### Example 3

### Comparative

### Oligomerization Test in the Presence of DEE

20 ml (12.8 g) of catalyst A in the form of grains (35-45 mesh) were charged into a fixed-bed tubular reactor with an inside diameter of 11 mm. Before the tests, the catalyst was activated at 550° C. under a stream of nitrogen for the 6 hours. After the activation, the reactor was cooled to 40° C. The catalyst was placed in contact with the charge at 40° C. under atmospheric pressure for 1 hour. Next, the pressure was increased to the reaction value and the reactor was heated to 200° C. at a rate of 30° C./hour. The temperature was maintained for 12 hours at 200° C. and was then increased up to 260° C. and 300° C. (30° C./hour).

The charge used for this oligomerization test is a fraction LLCCS containing 83% by weight of C5 hydrocarbons (of which 59% by weight are olefins and 41% by weight are paraffins).

The mixture comprising 85% by weight of LLCCS and 15% by weight of DEE was placed in contact with catalyst A under the following conditions:

reactor inlet temperature: 260, 300° C.

pressure P: 40 barg

hourly space velocity (pph): 1 h⁻¹.

Analysis of the product obtained was performed online by gas chromatography, the chromatograph being equipped with a capillary column.

On exiting the reactor, the gaseous phase, the liquid organic phase and the aqueous phase were separated. No recycling was performed.

The catalyst showed very rapid deactivation, which cannot be compensated for by increasing the temperature (FIG. 1).

The curve of simulated distillation measured for the liquid organic phase is reported in FIG. 2.

FIGS. 1-2 demonstrate the greatest deactivation and the highest temperature necessary in order to obtain complete conversion of C5 olefins in the presence of DEE. On the other hand, the preconversion of ethanol to butanol leads to a lower deactivation rate and a higher conversion into C5 olefins.

1. A process for producing distillate, hydrocarbons containing 10 or more carbon atoms, especially middle distillates, by conversion of a hydrocarbon-based charge containing C3-C10 olefins, comprising a first step of preconversion of light oxygenated molecules by condensation, these light oxygenated molecules comprising at least one alcohol comprising at least two carbon atoms, and a second step of oligomerization, in which the hydrocarbon-based charge is oligomerized in at least one oligomerization reactor in the presence of at least part of the effluent derived from the first step.

2. The process as claimed in claim 1, in which the alcohol comprising at least two carbon atoms, for example ethanol, is derived from biomass.

3. The process as claimed in claim 1, in which the light oxygenated molecules comprise, in addition to alcohol comprising at least two carbon atoms, oxygenated molecules containing one or two carbon atoms chosen from methanol, DME, DEE, formaldehyde, acetaldehyde and ethylene glycol, or mixtures thereof.

4. The process as claimed in claim 1, in which the first condensation step comprises the placing in contact, in aqueous phase, of light oxygenated molecules with at least one basic catalyst, at a temperature and a pressure that are sufficient to obtain a liquid effluent containing at least 40% by weight and preferably at least 50% by weight of C3+ molecules, and less than 10% by weight of ethylene.

5. The process as claimed in claim 1, in which the effluent derived from the first step is conveyed to a separation zone in order to extract therefrom, at least partly, the unreacted light oxygenated molecules, the ethylene and the other light gases.

6. The process as claimed in claim 1, in which the effluent derived from the first step is treated by selective hydrogenation in order to convert the aldehydes into heavier alcohols and the diolefins into olefins.

7. The process as claimed in claim 1, in which the first step is performed at a temperature of from 150 to 500° C. and at a pressure of from 0.1 to 40 MPa.

8. The process as claimed in claim 1, in which the catalyst used for the first step of the invention has a CO₂ adsorption capacity at 100° C. of from 0.03 to 10 mg/m² and preferably from 0.01 to 1 mg/m².

9. The process as claimed in claim 1, in which the catalyst used for the first step of the invention has a specific surface area of at least 10 m²/g, more specifically from 20 to 250 m²/g and preferably from 25 to 280 m²/g.

10. The process as claimed in claim 1, in which the total weight ratio between the effluent derived from the first step and the hydrocarbon-based charge is from 0.0001 to 1000 and preferably from 0.005 to 100.

11. The process as claimed in claim 1, in which, in the second step, all the effluent derived from the first step is added to the hydrocarbon-based charge.

12. The process as claimed in claim 1, in which all the amount of effluent to be added to the hydrocarbon-based charge is added thereto before it enters the oligomerization reactor(s).

13. The process as claimed in claim 1, in which part of the amount of effluent to be added to the hydrocarbon-based charge is added thereto before it enters the oligomerization reactor(s), the remaining part being added to the oligomerization reactor(s).

14. The process as claimed in claim 1, in which, during the second step, the hourly space velocity of the hydrocarbon-based charge is from 0.1 to 20 h⁻¹, preferably from 0.5 to 15 h⁻¹ and more preferably from 1 to 8 h⁻¹.

15. The process as claimed in claim 1, in which, during the second step, the pressure across the oligomerization reactor(s) is from 8 to 500 bara, preferably 10-150 bara and more preferably from 14 to 49 bara.

16. The process as claimed in claim 1, in which the effluent derived from the oligomerization step is conveyed to a separation unit in order to separate the fractions into C5-C9, C10-C12 and C12+.
